(19) United States
(12) Patent Application Publication
(10) Pub. No.: US 2020/0080089 A1
(43) Pub. Date: Mar. 12, 2020

(54) High-Throughput Aptamer Screening Assay

(71) Applicant: BellBrook Labs, Madison, WI (US)

(72) Inventors: Robert G. Lowery, Madison, WI (US); Aniket Aniket, Madison, WI (US)

(21) Appl. No.: 16/562,727

(22) Filed: Sep. 6, 2019

Related U.S. Application Data
(60) Provisional application No. 62/728,147, filed on Sep. 7, 2018.

(51) Int. Cl. C12N 15/115; G01N 21/64; G01N 33/542

(52) U.S. Cl. CPC C12N 15/115; G01N 21/6428; G01N 2021/6441; C12N 2310/16; G01N 33/542

(57) ABSTRACT

Methods and materials for development of high-throughput screening assays using aptamers are provided by this invention.

This application claims priority to U.S. Provisional Application Ser. No. 62/728,147, filed Sep. 7, 2018, which is incorporated by reference in its entirety.

### BACKGROUND OF THE INVENTION

### Field of invention

The present invention relates to methods and materials for development of high-throughput screening assays using aptamers.

### Description of Related Art

Neurodegenerative diseases represent an enormous unmet medical need, constituting more than $1.3 trillion in non-discretionary health care expenses in the US, which is expected to increase with the aging population. Existing treatments have little or no effect on the course of disease, and patients have to cope with the loss of brain and body function for the rest of their lives. Modulating the level of neurotrophic factors (NF) is a compelling therapeutic strategy as multiple lines of evidence indicate that they substantially improve neuronal vitality and function in neurodegenerative diseases. NFs are secreted soluble proteins that regulate growth, survival, and morphological plasticity of neurons (Loughlin, et al., 2012, Elsevier). These specialized growth factors have the ability to activate neuronal repair genes, particularly when exposed to supra-physiological levels (Bartus, et al., 2013, Neurobiology of aging, 34(1):35-61). Activation of these genes stimulates morphological and functional restoration of the degenerating neurons, significantly slowing further neurodegeneration and protecting against cell death (Loughlin, et al., 2012, Elsevier; Hefti, et al., 1989, Neurobiology of aging, 10(5)515-533). As such, modulation of NFs offers compelling opportunities in the treatment and management of a multitude of neurological diseases including Parkinson's, Alzheimer's, Huntington's, and amyotrophic lateral sclerosis (ALS).

Deficiencies in NFs are common in neurodegenerative diseases and very likely play a causative role in some of them. Currently, methods for detecting NFs, especially in a format compatible with automated HTS platforms, are not available for most NFs. The only HTS-compatible method currently available is Perkin Elmer's AlphaLisa, which is limited to glial cell-derived neurotrophic factor (GDNF), brain-derived neurotrophic factor (BDNF), and nerve growth factor (NGF). The method relies on fluorescence resonance energy transfer (FRET) between two bead-bound antibodies and singlet oxygen production to produce a chemiluminescent signal. This approach is expensive due to the cost of antibodies and specialized beads (Gale, et al., 2015), and is not amenable to multiplexing. Additionally, the donor beads are light sensitive, which complicates reagent and plate handling protocols. Moreover, the singlet oxygen species can be sequestered by compounds that scavenge radicals resulting in false signals (Eglen, et al., 2008, Current Chemical Genomics, 1:2). Other than AlphaLisa, researchers generally have to rely on ELISAs, which require multiple wash steps and are generally not used for automated HTS because of the complex plate and liquid handling required. Additionally, concerns have been raised about the lack of specificity in antibodies in general, which can lead to artifacts and inaccurate results (Pradidarcheep, et al., 2008, Journal of Histochemistry & Cytochemistry, 56(12):1099-1111; Michel, et al., 2009, Naunyn-Schmiedeberg's Archives of Pharmacology, 379(4):385-388). For instance, independent analyses of 49 commercial antibodies against 19 distinct GPCR-related receptors showed that none of the antibodies were selective (Bodei, et al., 2009, Naunyn-Schmiedeberg's Archives of Pharmacology, 379(4):413-415; Hamdani, et al., 2009, Naunyn-Schmiedeberg's Archives of Pharmacology, 379(4):403-407; Jositsch, et al., 2009, Naunyn-Schmiedeberg's Archives of Pharmacology, 379(4):389-395; Pradidarcheep, et al., 2009, Naunyn-Schmiedeberg's Archives of Pharmacology, 379(4):397-402; Jensen, et al., 2009, Naunyn-Schmiedeberg's Archives of Pharmacology, 379(4):409-412. Similarly, another study found that only one of four antibodies against programmed cell death ligand-1 fit selectivity criteria (Velcheti, et al., 2014, Laboratory Investigation, 94(1):107-116).

HTS-compatible assays are not available for most NFs, which is hampering efforts to target them therapeutically. Therefore, there remains a need to develop sensors conducive to performing HTS assays for NF detection.

### SUMMARY OF THE INVENTION

It is against the above background that the present invention provides certain advantages and advancements over the prior art.

Although this invention disclosed herein is not limited to specific advantages or functionalities, the invention provides a sensor for measuring an analyte, comprising:

(a) an aptamer and

(b) an oligonucleotide;

wherein the oligonucleotide is complementary to the aptamer;

wherein the aptamer and the oligonucleotide are individually conjugated to a lanthanide donor or an organic fluor acceptor; and

wherein the aptamer and the oligonucleotide are dissociated in the presence of the analyte.

The invention also provides a sensor for measuring an analyte, comprising:

(a) an aptamer and

(b) an oligonucleotide;

wherein the oligonucleotide is complementary to the aptamer;

wherein the aptamer and the oligonucleotide are individually conjugated to a lanthanide donor or an organic fluor acceptor; and

wherein the aptamer and the oligonucleotide are associated in the presence of the analyte.

The invention also provides a sensor for measuring an analyte, comprising:

(a) a first aptamer specific for a first analyte;

(b) a second aptamer specific for a second analyte;

(c) a first oligonucleotide; and

(d) a second oligonucleotide;

wherein the first oligonucleotide is complementary to the first aptamer;

wherein the second oligonucleotide is complementary to the second aptamer;

wherein the first and the second aptamer and the first and the second oligonucleotide are each individually conjugated to a lanthanide donor or an organic fluor acceptor;

wherein the first aptamer and the first oligonucleotide are dissociated in the presence of the first analyte; and

wherein the second aptamer and the second oligonucleotide are dissociated in the presence of the second analyte.

The invention also provides a sensor for measuring an analyte, comprising:

(a) a first aptamer specific for a first analyte;

(b) a second aptamer specific for a second analyte;

(c) a first oligonucleotide; and

(d) a second oligonucleotide;

wherein the first oligonucleotide is complementary to the first aptamer;

wherein the second oligonucleotide is complementary to the second aptamer;

wherein the first and the second aptamer and the first and the second oligonucleotide are each individually conjugated to a lanthanide donor or an organic fluor acceptor;

wherein the first aptamer and the first oligonucleotide are associated in the presence of the first analyte; and

wherein the second aptamer and the second oligonucleotide are associated in the presence of the second analyte.

The invention also provides a method for measuring an analyte, comprising:

(a) contacting a sensor with an analyte;

wherein the sensor comprises an aptamer associated with a complementary oligonucleotide in the absence of the analyte;

wherein the aptamer and oligonucleotide are individually conjugated to a lanthanide donor or an organic fluor acceptor; and

wherein the aptamer and oligonucleotide disassemble in the presence of the analyte; and

(b) detecting a signal generated upon dissociation of the aptamer and oligonucleotide; thereby measuring the analyte.

The invention also provides a method for measuring an analyte, comprising:

(a) contacting a sensor with an analyte;

wherein the sensor comprises an aptamer and an oligonucleotide;

wherein the oligonucleotide is complementary to the aptamer;

wherein the aptamer and oligonucleotide are individually conjugated to a lanthanide donor or an organic fluor acceptor; and

wherein the aptamer and oligonucleotide assemble in the presence of the analyte to form a trimeric complex comprising the aptamer, the oligonucleotide, and the analyte; and

(b) detecting a signal generated upon assembly of the trimeric complex; thereby measuring the analyte.

The invention also provides a method for measuring an analyte, comprising:

(a) contacting a sensor with an analyte;

wherein the sensor comprises a first aptamer specific for a first analyte associated with a first complementary oligonucleotide in the absence of the first analyte, and a second aptamer specific for a second analyte associated with a second complementary oligonucleotide in the absence of the second analyte;

wherein the first and the second aptamer and the first and the second oligonucleotide are each individually conjugated to a lanthanide donor or an organic fluor acceptor;

wherein the first aptamer and the first oligonucleotide disassemble in the presence of the first analyte;

wherein the second aptamer and the second oligonucleotide disassemble in the presence of the second analyte; and

(b) detecting a first signal generated upon dissociation of the first aptamer and the first oligonucleotide; thereby measuring the first analyte; and

(c) detecting a second signal generated upon dissociation of the second aptamer and the second oligonucleotide; thereby measuring the second analyte.

The invention also provides a method for measuring an analyte, comprising:

(a) contacting a sensor with an analyte;

wherein the sensor comprises:
(i) a first aptamer specific for a first analyte;
(ii) a second aptamer specific for a second analyte;
(iii) a first oligonucleotide; and
(iv) a second oligonucleotide;
wherein the first oligonucleotide is complementary to the first aptamer;
wherein the second oligonucleotide is complementary to the second aptamer;
wherein the first and the second aptamer and the first and the second oligonucleotide are each individually conjugated to a lanthanide donor or an organic fluor acceptor;
wherein the first aptamer and the first oligonucleotide assemble in the presence of the first analyte to form a first trimeric complex comprising the first aptamer, the first oligonucleotide, and the first analyte;
wherein the second aptamer and the second oligonucleotide assemble in the presence of the second analyte to form a second trimeric complex comprising the second aptamer, the second oligonucleotide, and the second analyte;

(b) detecting a first signal generated upon assembly of the first trimeric complex; thereby measuring the first analyte; and

(c) detecting a second signal generated upon assembly of the second trimeric complex; thereby measuring the second analyte.

In one aspect of the methods disclosed herein, the signal is a time-resolved fluorescence energy transfer (TR-FRET) signal.

In one aspect of the sensors and methods disclosed herein, the lanthanide donor is a terbium, europium, and/or samarium chelate.

In one aspect of the sensors and methods disclosed herein, the organic fluor acceptor is Fluorescein, rhodamine, Texas Red, an Alexa Fluor, a Cyanine dye, and/or an Atto dye.

In one aspect of the sensors and methods disclosed herein, the organic fluor acceptor is a non-overlapping organic fluor acceptor.

In one aspect of the sensors and methods disclosed herein, the analyte is an amino acid, an amino acid derivative, a peptide, a protein, a steroid, a lipid, a sugar, a carbohydrate, a drug molecule, a drug metabolite, a coenzyme, a nucleotide, a nucleotide derivative, a cyclic nucleotide, and/or a cyclic dinucleotide.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The following detailed description of the embodiments of the present invention can be best understood when read in conjunction with the following drawings, where like structure is indicated with like reference numerals and in which:

FIG. 1A-1B show various configurations for aptamer-based sensors. FIG. 1A is a schematic of a structure switching oligo dissociation sensor, and FIG. 1B is a schematic of a structure switching oligo association sensor.

FIG. 2 is a schematic of the AptaFluor time-resolved fluorescence energy transfer (TR-FRET) assay. TR-FRET between Eu-chelate (Eu) on aptamer and Dylight-650 fluor (F) on a complimentary oligo (C-oligo) hybridized to the aptamer is disrupted by VEGF binding. Change in aptamer's conformation forces C-oligo away from the aptamer. See Example 3.

FIG. 3A shows binding curves for TR-FRET assay in DMEM media showing dose dependence and signal stability over time.

FIG. 3B shows statistical analysis shows rapid attainment of high Z′ value indicating assay robustness.

FIG. 3C shows that no cross-reactivity was observed between VEGF and other common growth factors.

FIG. 3D shows multiplexed detection of VEGF and SAH using Tb-Chelate donor and Alexa-488 and Dylight-650 as acceptor fluors. See Example 1.

FIG. 4 shows emission spectra of Tb-chelate. See Example 4.

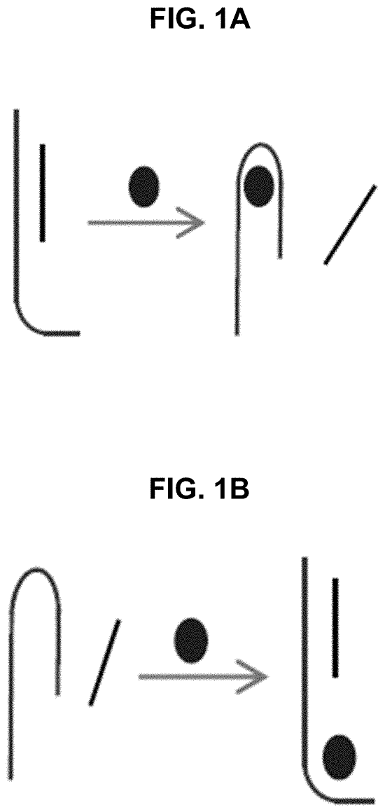

Skilled artisans will appreciate that elements in the figures are illustrated for simplicity and clarity and have not necessarily been drawn to scale. For example, the dimensions of some of the elements in the figures can be exaggerated relative to other elements to help improve understanding of the embodiment(s) of the present invention.

### DETAILED DESCRIPTION OF THE INVENTION

All publications, patents and patent applications cited herein are hereby expressly incorporated by reference for all purposes.

Before describing the present invention in detail, a number of terms will be defined. As used herein, the singular forms “a”, “an”, and “the” include plural referents unless the context clearly dictates otherwise. For example, reference to a “nucleic acid” means one or more nucleic acids and reference to “an analyte” means one or more analyte.

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

As used herein, the terms “polynucleotide,” “nucleotide,” “oligonucleotide,” and “nucleic acid” can be used interchangeably to refer to nucleic acid comprising DNA, RNA, derivatives thereof, or combinations thereof.

As used herein, the terms “homogenous assay,” “homogenous format,” and “homogenous detection” can be used to refer to detection of an analyte by a simple mix and read procedure. A homogenous assay does not require steps such as sample washing or sample separation steps. Examples of homogenous assays include TR-FRET, FP, FI, and luminescence-based assays.

As used herein, the term “and/or” is utilized to describe multiple components in combination or exclusive of one another. For example, “x, y, and/or z” can refer to “x” alone, “y” alone, “z” alone, “x, y, and z,” “(x and y) or z,” “x and (y or z),” or “x or y or z.”

### Aptamers and Aptamer-Based Sensors

Aptamers are nucleic acid affinity reagents that have been developed for detection of diverse ligands ranging in size from small molecules (cocaine, thalidomide, ATP, dopamine) to proteins (VEGF, EGFR) to cells (McKeague & Derosa, 2012, J Nucleic Acids 2012:748913; Yuce et al., 2015, Analyst 140(16):5379-99). As used herein, the term “aptamer” can be used to refer to a molecule that can bind to a specific target with high specificity and affinity. The aptamer can be an oligonucleotide, such as DNA or RNA, or a peptide. In particular, the aptamer can be a single-stranded oligonucleotide, such as single-stranded DNA. DNA and RNA aptamers can exhibit subnanomolar affinity and exquisite selectivity for their ligands (McKeague & Derosa, 2012, J Nucleic Acids 2012:748913; Yuce et al., 2015, Analyst 140(16):5379-99) and are thus well suited for detecting analytes in complex mixtures like cell lysates or serum. Moreover, evidence suggests that aptamers are more specific than antibodies for small or structurally subtle epitopes such as methyl and acetyl moieties. For example, a well characterized RNA aptamer for theophylline discriminates against caffeine with more than 10⁴-fold selectivity on the basis of a single methyl group (McKeague & Derosa, 2012, J Nucleic Acids 2012:748913).

The aptamers utilized herein can be conjugated to a dye, such as an organic donor fluor or an organic acceptor fluor, a luminescent lanthanide, or a peptide. The peptide can be a luciferase peptide. A luminescent lanthanide can be attached to an aptamer by interactions not limited to a streptavidin-biotin interaction.

As used herein, the term “split aptamer” can be used to refer to an aptamer that is composed of two or more fragments. For example, a split aptamer can be composed of two fragments, i.e. P1 and P2. Split aptamers retain specificity for their targets (Sharma et al., 2011, J Am. Chem. Soc. 133(32):12426-9) and have been shown to recognize a variety of molecules such as thrombin (Chen et al., 2010, Biosensors and Bioelectronics 25(5):996-1000), adenosine (Yang et al., 2011, Analytical Methods 3(1):59-61 and Wang et al., 2011, Sensors and Actuators B: Chemical 156(2):893-8), ATP (Liu et al., 2010, Chemistry-A European Journal 16(45):13356-9 and He et al., 2013, Talanta 111:105-110), and cocaine (Sharma et al., 2012, Analytical Chemistry 84(14):6104-9) and may also improve the detection sensitivity as compared to intact aptamers (Liu et al., 2014, Sci. Rep. 4:7571). Moreover, the length of an intact aptamer is not thought to be limiting factor, since split aptamers generated from relatively short 15-mer thrombin or 27-mer ATP aptamer are capable of self-assembly (Chen & Zeng, 2013, Biosensors and Bioelectronics 42:93-99). Typically, a full-length aptamer is split into two parts in a way such that the target molecule bound in the pocket forms a bridge between the two split fragments.

As used herein, the term “riboswitch” can be used to refer to a structured noncoding RNA molecule capable of binding to an analyte and/or regulating gene expression. As used herein, riboswitches are microbial metabolite sensing RNA aptamers. Riboswitches, which are naturally occurring aptamers, have been discovered for diverse biomolecules including sugars, amino acids, and cyclic nucleotides (Breaker, 2012, Cold Spring Harb. Perspect. Biol. 4(2):a003566. Riboswitches can discriminate with more than 1000-fold selectivity on the basis of a single methyl group; e.g., for S-adenosylhomocysteine (SAH) versus S-adenosylmethionine (SAM) (Wang et al., 2008, Mol Cell. 29(6):691-702).

### Aptamers as Affinity Reagents for Homogenous Detection of Soluble Factors

Nucleic acid aptamers exhibit sub-nanomolar affinity and exquisite selectivity, and are well suited for applications based on molecular recognition as analytical, diagnostic and therapeutic tools (Tombelli, et al., 2005, Biosensors and Bioelectronics, 20(12):2424-2434). They are generated using an in vitro selection process called SELEX (Systematic Evolution of Ligands by EXponential enrichment), which is a key advantage over the lengthy in vivo methods used to generate antibodies (Yüce et al., 2015, Analyst, 140:5379-5399; Stoltenburg, et al., 2007, Biomolecular Engineering, 24(4):381-403; Jayasena, 1999, Clinical Chemistry, 45(9):1628-1650). Moreover, evidence suggests that they can be more specific than antibodies for discriminating between structurally subtle epitopes (McKeague, et al., 2012, Journal of Nucleic Acids, 2012). Indeed, minimizing cross-reactivity is critical for NF detection assays, given the high degree of structural similarity between NGF and several NFs, particularly BDNF, which shares ˜50%, sequence homology with NGF (Ernfors, et al., 1990, Proceedings of the National Academy of Sciences, 87(14):5454-5458). Moreover, SELEX enables counter-screening against molecules that contribute to cross-reactivity and interference. Aside from their favorable properties as affinity reagents aptamers can be engineered to undergo ligand-dependent conformational shifts that can be transduced to produce FRET-based signals (Li, et al., 2010, Accounts of Chemical Research, 43(5):631-641; Nutiu, et al., 2003, Journal of the American Chemical Society, 125(16):4771-4778; Nuiti, et al., 2005, Methods, 37(1):16-25).

The affinity and specificity of aptamers can be further enhanced following the initial selection using rapid in vitro methods such as site directed mutagenesis and directed evolution, an approach that was recently used to increase the selectivity of a histone H4 aptamer more than 20-fold (Yu et al., 2011, Chembiochem. 12(17):2659-66). Aptamers are also less expensive to produce and have lower batch-to-batch variation compared to antibodies. In addition, they are much easier to engineer and modify in specific ways than antibodies, such as incorporation of fluorophores at specific sites, because most desired changes can be introduced during solid state synthesis (Juskowiak, 2011, Anal. Bioanal. Chem. 399(9):3157-76). In contrast, specific labeling of antibodies often requires insertion of non-native amino acids, which is extremely time consuming and requires specialized expertise, and the results are difficult to predict (Sochaj et al., 2015, Biotechnol. Adv. 33(6 Pt 1):775-84).

To be practically useful in biomedical research applications, an aptamer based assay must be useful in high throughput applications such as screening chemical libraries for potential drug molecules or testing large numbers of biological samples for the presence of disease biomarkers (Kong et al., 2012, J. Lab. Autom. 17(3):169-85; Nicolaides et al., 2014, Front. Oncol. 4:141; and Hughes et al., 2011, Br. J. Pharmacology 162(6):1239-49). Utility for HTS applications imposes strict requirements on aptamer based assays, most notably that they are configured in a homogenous or “mix-and-read” format and that they produce a signal that provides sensitive detection with minimal interference using instruments commonly found in HTS laboratories (Hughes et al., 2011, Br. J. Pharmacology 162(6):1239-49 and Jones et al., 2004, Assay Guidance Manual, Ed. Sittampalam et al.).

One of the main advantages of aptamers over antibodies for detection is that the signaling component can be integrated into the aptamer itself to produce a sensor. One of the main advantages of sensors is that they provide direct detection of analytes without the use of additional detection reagents. This inherent simplicity is advantageous both from an assay development standpoint and for practical use. Sensors do not require the development of additional reagents, such as a second aptamer for solid phase assay or a tracer for competitive assays. In addition, aptamer-based sensors are generally formatted for homogenous detection, which makes them well suited for HTS. This is especially advantageous for molecules that are too small to accommodate binding of two antibodies for a sandwich assay format. Detection of these small molecules with antibodies requires competitive assays, such as competitive ELISAs, or radioimmunoassays (RIAs). The use of RIAs is highly undesirable due to radiation hazards and the associated regulatory and disposal costs. Competitive assays are undesirable because they generally produce a negative signal, and thus are not as sensitive and have a limited dynamic range.

An exemplary signaling mechanism used for aptamer-based sensors is a change in the properties of an attached fluor upon analyte binding. Ligand binding often induces structural shifts in the aptamer which can change the microenvironment of attached fluors resulting in quenching, enhanced emission, or changes in polarization. For example, for aptamers that bind proteins, a fluor attached to the aptamer usually undergoes an increase in polarization upon formation of the protein-aptamer complex because of the slower rotational mobility of the complex relative to the free aptamer. Alternatively, a fluor can be attached to a complementary oligonucleotide that undergoes displacement or, less commonly, annealing, due to ligand-induced structural shifts in the aptamer. A common configuration for such oligo-displacement assays is to attach a quencher to one element (i.e., the aptamer or the complementary oligo) and a fluor to the other, such that ligand induced dissociation of the oligo results in enhanced fluorescence. For reasons that are not fully understood, it is sometimes not possible to produce robust aptamer based sensors using a structure switching approach. Though efforts in this direction are ongoing, the difficulty in developing structure switching aptamers remains a significant hurdle in development of aptamer based sensors.

### Neurodegenerative Diseases and NFs

Parkinson's disease (PD), which affects approximately one million people in the U.S. alone and more than 10 million worldwide, is caused due to progressive degeneration of nigrostriatal dopamine (DA) neurons resulting in debilitating loss of motor functions. A growing body of evidence indicates the usefulness and efficacy of NFs for the treatment of PD (Kells, et al., 2012, Neurobiology of disease, 48(2):228-235; Yasuda, et al., 2010, Expert review of neurotherapeutics, 10(6):915-924; Emborg, et al., 2009, Neurobiology of disease, 36(2):303-311; Zhao, et al., 2014, PloS one, 9(9):e106867).

One of the most well documented NF to alleviate PD symptoms is glial cell line-derived neurotrophic factor (GDNF), which exerts neuroprotective effect on DA neurons to promote survival and stimulate neurite growth (Hefti, et al., 1989, Neurobiology of aging, 10(5)515-533; Eggert, et al., 1999, Neuroscience letters, 269(3):178-182; Hegarty, et al., 2014, Molecular neurobiology, 50(2):559-573). GDNF has also been shown to regenerate the nigrostriatal DA pathway in cell and animal models of PD (Hegarty, et al., 2014, Neural regeneration research, 9(19):1708; Nam, et al., Molecular neurobiology, 2015, 51(2):487-499). GDNF modulates DA neuronal excitability via changes in transient A-type K⁺ channels (Yang, et al., 2001, Nature neuroscience, 4(11):1071-1078) that can in-turn increase DA release (Hebert, et al., 1996, Journal of Pharmacology and Experimental Therapeutics, 279(3):1181-1190; Peterson, et al., 2008, Neurotherapeutics, 5(2):270-280). In rats, dopaminergic neuron degeneration induced by 6-hydroxydopamine injections resulted in a loss of about 50-80% of the nigral DA neurons leading to progressive cellular degeneration. Repeated GDNF doses promoted survival of the remaining nigral DA cells, and once rescued, they survived for months without GDNF dosing, indicating the lasting effect of GDNF on survival (Rosenblad, et al., 2000, Experimental neurology, 161(2):503-516; Winkler, et al., 1996, The Journal of neuroscience, 16(22):7206-7215; Sauer, et al., 1994, Neuroscience, 59(2):401-415).

Increasing evidence suggests that synaptic dysfunction is a key pathophysiological hallmark in many neurodegenerative disorders (Yoshii, et al., 2010, Developmental neurobiology, 70(5):304-322; Lu, et al., 2013, Nature Reviews Neuroscience, 14(6):401-416). Brain-derived neurotrophic factor (BDNF) has powerful synaptic effects in promoting synaptic transmission, plasticity, and BDNF-based synaptic repair has been targeted a disease-modifying strategy for neurodegenerative diseases, such as Alzheimer's disease, where a decrease in number of synapse correlates with disease progression (Selkoe, D J, 2002, Science, 298(5594):789-791; Terry, et al., 1991, Annals of neurology, 30(4):572-580). Overall, it is thought that BDNF promotes synapse formation by regulating axonal branching, dendritic growth, and activity-dependent synapse refinement (Park, et al., 2013, Nature Reviews Neuroscience, 14(1):7-23; McAllister, et al., 1995, Neuron, 15(4):791-803; Cabelli, et al., 1995, Science, 267(5204):1662). Mice lacking BDNF receptor, TrkB, exhibit a decrease in the number of total synaptic vesicles per synapse, as well as overall synapse number (Pozzo-Miller, et al., 1999, The Journal of Neuroscience, 19(12):4972-4983; Genoud, et al., 2004, The journal of neuroscience, 24(10):2394-2400). Acute application of BDNF has been found to rapidly enhance synaptic transmission and transmitter release, and mediate increased synapse sprouting (Jovanovic, et al., 2000, Nature neuroscience, 3(4):323-329). BDNF is required for the establishment of proper number of dopaminergic neurons in the substantia nigra (Baquet, et al., 2005, The Journal of neuroscience, 25(26):6251-6259), and post-mortem analyses of PD patient brains have shown reduced levels of BDNF and BDNF mRNA suggesting that the lack of this NF may be involved in disease etiology and pathogenesis (Howells, et al., 2000, Experimental neurology, 166(1):127-135; Mogi, et al., 1999, Neuroscience letters, 270(1):45-48.

In the peripheral nervous system, the dominant NF is nerve growth factor (NGF), which acts on sympathetic and sensory neurons. Reduced NGF levels in heterozygous mice result in significant deficits in memory acquisition and retention (Chen, et al., 1997, The Journal of neuroscience, 17(19):7288-7296). Together with BDNF, NGF is essential for multiple functions throughout adulthood such as memory acquisition and retention, long-term potentiation, and cholinergic innervation (Chen, et al., 1997, The Journal of neuroscience, 17(19):7288-7296; Weissmiller, et al., 2012, Translational neurodegeneration, 1(1):1). Animal models of AD and Down syndrome show decreased NGF levels associated with basal forebrain cholinergic neurons (BFCN), indicating NGF's strong role in preventing cellular death of BFCN (Cooper, et al., 2001, Proceedings of the National Academy of Sciences, 98(18):10439-10444; Venero, et al., 1994, Neuroscience, 59(4):797-815). Although clinical trials with some NFs, such as GDNF, have produced mixed results, there is broad agreement that these setbacks are not due to NFs per se but due to inappropriate dosing and lack of proper drug delivery strategy in effectively delivering proteins across the blood brain barrier. Enhancing NF expression with small molecules is a promising alternative in overcoming some of the unfavorable pharmacological properties (Massa, et al., 2010, The Journal of clinical investigation, 120(5):503-506; Longo, et al., 2007, Current Alzheimer Research, 4(5):503-506). Discovery of small molecule NF modulators will require robust methods for detecting NFs that are compatible with HTS platforms. Moreover, using NFs as biomarkers, whether for drugs that target them directly or as surrogate markers (Sen, et al., 2008, Biological Psychiatry, 64(6):527-532; Liu H-T, et al., 2008, The Journal of Urology, 179(6): 2270-2274) will require assays that can be used on automated high-throughput platforms.

Therefore, a full-spectrum analysis of NFs efficacy warrants a multi-factorial assessment of receptor/ligand interactions using phenotypic screening approaches. NFs and their associated diseases are shown in Table 1 (see e.g., Weissmiller, et al., 2012, Translational neurodegeneration, 1(1):1).

TABLE 1
NFs studied for disease treatment.

| Disease | NF | Status |
|---|---|---|
| ALS | NGF and BDNF | Recruiting |
| Parkinson's disease | GDNF/neurturin | Phase 1 complete, |
| | | Phase 2 ongoing |
| Huntington's disease | BDNF | Pre-clinical |
| Alzheimer's disease | NGF and BDNF | Phase 1 ongoing |
| Down syndrome | NGF | Pre-clinical |
| Spinal cord injury | BDNF and NT-3 | Pre-clinical |
| Sensory neuropathies | NGF | Phase 2 completed |
| Supranuclear palsy | GDNF | Phase 2 completed |

### Aptamers as Affinity Reagents for Homogenous Detection of Soluble Factors

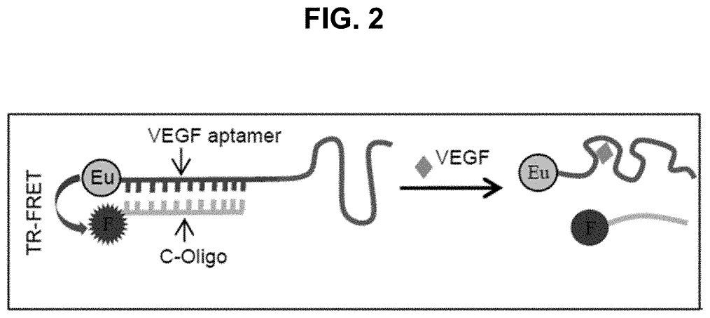

Aptamer sensors described herein are structure switching aptamers. The key functional feature of AptaFluor is the coupling of FRET between a lanthanide and a fluor with a ligand-induced conformational change in the aptamer. For example, the aptamer sensors can be structure dissociation aptamers. There are 4 design components (FIG. 2): (i) a lanthanide as long-lifetime energy donor, (ii) an aptamer as the bio-recognition molecule, (iii) a short oligonucleotide complimentary (C-oligo) to the aptamer, and (iv) an acceptor fluor. The fluor is chosen such that the emission wavelength of the lanthanide corresponds with the excitation wavelength of the fluor. In the absence of the ligand, the C-oligo hybridizes with the aptamer, resulting in a TR-FRET signal. Binding of the analyte induces a conformational change that disrupts C-oligo/aptamer duplex, thus creating sufficient separation to relieve FRET and reduce fluor emission.

Aside from their favorable properties as affinity reagents, aptamers have been previously engineered to undergo substantial conformational shifts on ligand binding that can be transduced to produce FRET-based signals (Li, et al., 2010, Accounts of Chemical Research, 43(5):631-641; Nutiu, et al., 2003, Journal of the American Chemical Society, 125(16):4771-4778; Nuiti, et al., 2005, Methods, 37(1):16-25); however, none of the existing aptamers have not been adopted for HTS largely because the signaling mechanisms used are not compatible with existing HTS instrumentation.

To overcome the aforementioned problems, the intramolecular switch properties of aptamers with time-gated luminescence properties of lanthanides have been leveraged, as disclosed herein. Background fluorescence from screening compounds, plastics, and biological specimens limits the utility of fluorescence intensity as a detection mode for HTS and diagnostics. On the other hand, lanthanide complexes, e.g., Tb, Eu and Ds, have long lifetimes in the range of 0.2 to 1.5 msec (Handl, et al., 2005, Life sciences, 77(4):361-371) compared to organic fluors (10 nsec), so when they are used as FRET donors, measurement of emission from the acceptor can be delayed until background fluorescence has decayed. In Time Resolved FRET (TR-FRET), the time delayed lanthanide emission is coupled to the excitation of an organic fluor; thus, the emission intensity of the fluor is a “clean” signal. Over the last ten years, lanthanide-based TR-FRET has become one of the most frequently used detection modes for HTS, and multimode readers have been optimized specifically for it. To enable the use of lanthanide-based TR-FRET with aptamers, an oligonucleotide displacement feature was engineered into a structure switching DNA aptamer against vascular endothelial growth factor (VEGF) to produce a ligand-dependent change in TR-FRET between a lanthanide (Eu-chelate or Tb-chelate) and an acceptor fluor (Dylight-650) (FIG. 2). VEGF binding to the aptamer displaces a short complimentary oligo (C-oligo) and interrupts TR-FRET signal; the decrease in signal is correlated to the protein concentration.

The aptamer sensors and methods described herein can be used to detect biomolecules not limited to NFs. For example, the structure switching aptamers disclosed herein can be used to detect amino acids and amino acid related molecules such as dopamine and thyroxine, peptides and proteins, steroids, lipids, sugars and carbohydrates, drug molecules and their metabolites, coenzymes such as acetyl-coenzyme A and cobalamin, nucleotides and nucleotide-related molecules such as nucleotide nucleotide-diphospho-sugars, pyridine nucleotides (NAD and NADH), cyclic nucleotides and cyclic dinucleotides. In some embodiments, the lanthanide donors disclosed herein can be a terbium, europium, and samarium chelate. In some embodiments, the organic fluor acceptors disclosed herein can be Fluorescein, rhodamine, Texas Red, Alexa Fluors such as AlexaFluor 633 and AlexaFluor 647, Cyanine dyes such as Cy3 and Cy5, Atto dyes such as Atto 594 and Atto 633.

In some embodiments, the novel signaling mechanism disclosed herein can be used to develop aptamer biosensors for multiplexed detection of NFs, using BDNF, NGF, and GDNF as targets. The AptaFluor NF platform can provide the following advantages over existing immunochemical methods: (1) picomolar detection and large dynamic range compared to competitive binding assays; (2) homogenous assay methods with no separation or wash steps, which allows for non-invasive, live cell assays and integration with automated workflows; (3) capacity for multiplex analysis to help delineate the role of NFs in neurodegenerative diseases and enable multi-parametric HTS assays; and (4) simplicity of reagents, production methods, and consumables to lower the cost of HTS assays.

The novel NF assays using TR-FRET based signaling mechanism described herein overcome a significant technical hurdle preventing basic research and drug discovery targeting NFs in neurodegenerative diseases. Moreover, the NF assays are part of a platform for the first exploitation of the exquisite affinity and selectivity of aptamers for commercially viable HTS assays. The reduced timelines and cost in developing aptamer-based assays compared to antibodies can enable rapid and economic development of a diverse AptaFluor toolbox for a wide range of targets with applications in basic research, drug discovery and companion diagnostics. The key applications of AptaFluor NF assay encompass: (1) phenotypic HTS assays that use one or more NFs as an endpoint; e.g., screening for small molecules that enhance expression of a target NF; (2) facile detection of NFs in cellular and animal models for basic research exploring their role in neurodegenerative diseases; and (3) detection of NFs in animal and clinical samples; i.e., biomarkers for disease status and/or prognosis.

In some embodiments, AptaFluor technology is expanded to additional neurotrophic factors including neurotrophin-3 and -4, artemin, neurturin, persephin, and ephrins, all of which are implicated in neurodegenerative diseases. Sensitivity, if necessary, can be increased using further in vitro selection and/or modified bases. In some embodiments, optimization of assays for detection in biological fluids to extend the platform to animal models takes place, as a stepping-stone for detection in clinical samples. Furthermore, the SELEX process can be refined and streamlines for rapid aptamer selection with higher specificity and sensitivity.

The invention will be further described in the following examples, which do not limit the scope of the invention described in the claims.

### EXAMPLES

The Examples that follow are illustrative of specific embodiments of the invention, and various uses thereof. They are set forth for explanatory purposes only, and are not to be taken as limiting the invention.

### Example 1

### Detection of VEGF

An aptamer for VEGF (Freeman et al., 2012, Analytical Chemistry 84(14):6192-8) was modified with a biotin group at its 5′ end. Dylight-650 fluor was covalently attached to the 3′ end of several C-oligos varying 9-13 bases in length that were complimentary to the 5′ end of the aptamer, and the biotinylated aptamer/C-oligo hybrid was conjugated to streptavidin-coated Eu-chelate. Binding isotherm data showed that a 10 base long C-oligo was required for efficient TR-FRET and sensor stability.

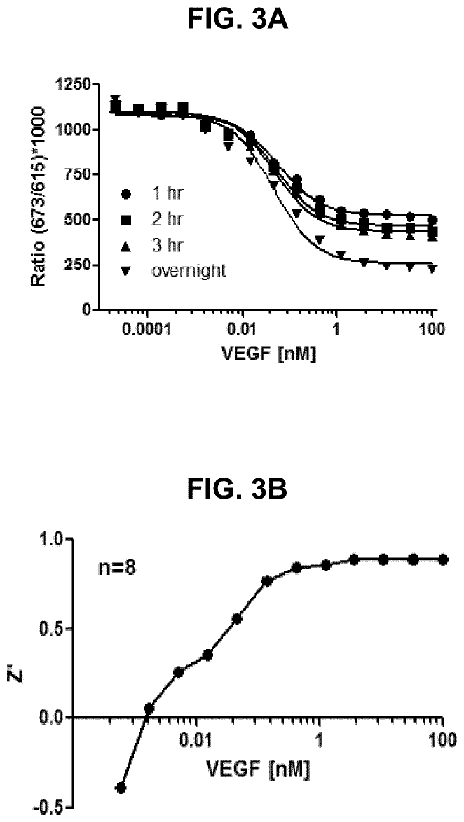

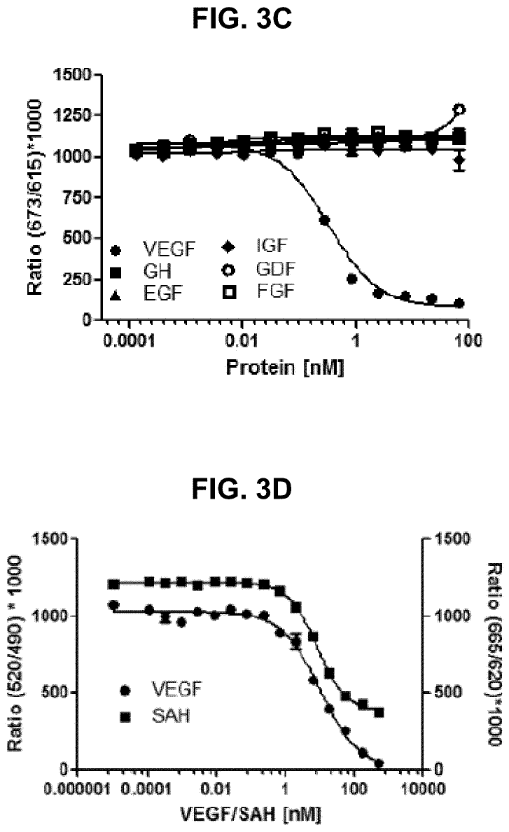

A binding isotherm in DMEM media with varying VEGF concentrations shows that the assay is extremely sensitive, with EC₅₀˜50 pM (FIG. 3A), which is similar to that obtained using a commercial ELISA assay (R&D Systems). The signal develops rapidly within 1 hr, and is quite stable even after overnight incubation. Assay quality assessment using the Z′ parameter, which reflects signal dynamic range and variability, shows Z′>0.5 at 20 pM and Z′>0.8 at 50 pM VEGF with a lower limit of detection (LLD) of 3 pM (FIG. 3B), indicating a robust assay with sufficient signal magnitude and precision for HTS. In vitro tests using primary HUVEC cells showed the ability of the sensor to detect low pM levels of cell-secreted VEGF. Binding specificity tested against an array of common growth factors showed no discernible cross-reactivity (FIG. 3C).

Two kinds of lanthanides, Eu-chelate and Tb-chelate, were tested, and greater sensitivity was found using Eu-chelate. Tb-Chelate, however, has multiple emission peaks, and using a combination of two aptamers, one for VEGF and other for small molecule S-adenosylmethionine (SAH), with two non-overlapping fluors (Alexa-488 and Dylight-650), multiplexed detection of both analytes was achieved by reading signals from two separate TR-FRET events (FIG. 3D).

### Example 2

### Selection and Characterization of Aptamers against GDNF, BDNF, and NGF

The conventional SELEX process for aptamer selection involves incubating a large random nucleotide library (10¹⁵ sequences) with an immobilized protein target, followed by separation of non-binding sequences. The bound sequences are PCR amplified and subjected to additional selection cycles (Yüce et al., 2015, Analyst, 140:5379-5399).

A DNA library comprising 10¹⁵ random sequences is subjected to repeated selection cycles for 10-12 rounds to yield a handful of sequences capable of binding to the target NF. A negative selection round after each positive selection round is carried out to increase selectivity by screening against non-target NFs. Positive selections using 1 mM target NF and negative selections using 0.1 mM non-target NFs are carried out, and target NF is gradually decreased to 1 μM and non-target NFs to 1 mM, to increase selection pressure for sequences having high binding affinity and selectivity.

All selection steps are carried out in cell culture media (Dulbecco's Modified Eagle's medium (DMEM)+10% fetal bovine serum (FBS)). High-throughput sequencing of libraries from each selection round are used to measure the enrichment of the aptamer sequence compared to its abundance in the starting library. These sequences are analyzed for statistical significance in terms of motif frequencies (deviation from random expectations) and copy number of entire sequences to guide in selection of aptamers. Finally, binding assays are performed and K_{d} values are determined using surface plasmon resonance (SPR) and validated aptamer function using commercially available antibodies (R&D Systems).

Successful generation of aptamers against all NFs with K_{d} of at least 10 nM and >250-fold specificity against other NFs is expected. Because aptamer selection has been carried out in cell culture media, these aptamers are expected to be immune to interference from media components. The target of selecting aptamers with K_{d}≤10 nM is reasonable based upon other structure-switching aptamers that have been reported (Li, et al., 2010, Accounts of Chemical Research, 43(5):631-641; Nutiu, et al., 2003, Journal of the American Chemical Society, 125(16):4771-4778; Oh, et al., 2010, Proceedings of the National Academy of Sciences, 107(32):14053-14058; Zhang, et al., 2008, Analytical Chemistry, 80(22):8382-8388), so it is anticipated that the aptamers described herein have the desired sensitivity and selectivity. If the library members bound to the 8-mer strands are not capable of distinguishing between the target NF and counter-selection analytes in the early rounds of selection, or some library members fold into stable secondary structures after pre-selection, thereby preventing their binding to the analyte, factors such as analyte concentration, selection buffer, and incubation time are optimized.

### Example 3

### Development of Independent TR-FRET Assay for NFs Using Lanthanide and Structure Switching Aptamers

The candidate aptamer(s) generated by SELEX are synthesized with a biotin moiety attached at the 5′ terminus via a C6 spacer and provided in purified form (Integrated DNA Technologies, Coralville, Iowa). Tb-chelate (lanthanide) coated with streptavidin (Thermo-Fisher, Waltham, Mass.) is attached non-covalently to the aptamer. Several C-oligos complimentary to the 5′ end of the aptamer(s) are evaluated to identify candidates that can (a) form stable duplexes with the aptamer; (b) facilitate efficient TR-FRET; and (c) undergo rapid displacement upon ligand binding. For each aptamer, C-oligos based on the position of specific 8-mer sequences (used during aptamer selection) are designed, since these would be complimentary to the structure-switching region of the aptamer. C-oligos 9-13 bases in length are tested (Li, et al., 2010, Accounts of Chemical Research, 43(5):631-641; Nuiti, et al., 2005, Methods, 37(1): 16-25; Lu, et al., 2008, Analytical chemistry, 80(6):1883-1890. The C-oligos are synthesized with Dylight-650 attached to the 3′ end via C6 spacer. The most promising C-oligo for each aptamer are characterized in detail by analyzing the kinetics and equilibrium of ligand binding and signal stability. While the conditions used for aptamer selection provide some basis for optimal buffer conditions, buffer and salt type, pH, and ionic strength are re-optimized to enhance assay sensitivity and S/B ratio. Binding specificity is tested against an array of other NFs and growth factors. In addition, the stability of the AptaFluor is tested for 24-48 hours at 37° C. in cell culture media. All analyses are performed in aqueous buffer as well as in cell culture media in 384 well plates using the M-1000 plate reader (Tecan, Männedorf, Switzerland).

Development of three independent assays is expected, which detect each of the NFs at concentrations of 1 nM or less, S/B ratio ≥3, LLD ≤0.1 nM, and at-least 250-fold specificity as compared to other NFs. The sensor(s) stability is expected to be ≥24 h at 37° C. in cell culture media. If a high enough assay window is not achieved, the configuration is reversed and Tb-chelate is attached at the 3′ end (as opposed to attaching the lanthanide at the 5′ end of the aptamer).

### Example 4

### Design of Multiplexed TR-FRET Assay for Simultaneous Detection of NFs

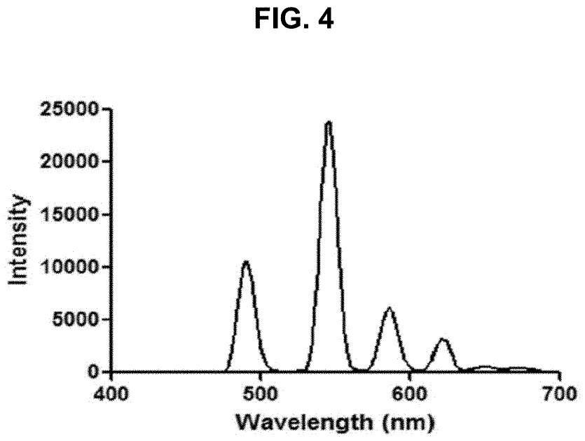

A significant improvement in existing HTS workflows would be the ability to simultaneously detect multiple analytes, such as applications involving screening for on-target/off-target effects within the same sample. The emission spectrum of Tb-chelate is characterized by 4 sharp peaks at 490, 546, 583 and 620 nm (FIG. 4).

A multiplexed TR-FRET assay using 3 of these peaks can be used for simultaneous detection of GDNF, BDNF, and NGF. In this configuration, Tb-Chelate is excited at a single wavelength in the UV range (340 nm), and its concomitant emission spectra is coupled to 3 different acceptor fluors on C-oligos (for each NF aptamer). The C-oligos for each of the NF are conjugated separately to three distinct, judiciously selected fluors that exhibit narrow, non-overlapping emission peak and are FRET-capable with Tb-chelate. Furthermore, the time-resolved nature of these measurements and the large Stokes shift of Tb-chelate would significantly reduce background and spectral cross-talk by preventing direct excitation of the acceptor fluors (Handl, et al., 2005, Life sciences, 77(4):361-371).

AlexaFluor 488 (Ex-495 nm/Em-520 nm), BODIPY TMR (Ex-542 nm/Em-574 nm), and Dylight 650 (Ex-652 nm/Em-672 nm) are conjugated on the three C-oligos corresponding to each NF. Such a configuration allows each fluor to engage in FRET with Tb-chelate independently, as their excitation peak corresponds to 490, 546, 620 nm emission peak of Tb-chelate. Fluorescence bleed-through can be determined by measuring the intensity of each fluor in the presence and absence of fixed concentration of the other two fluors. For TR-FRET measurements, the emission bandwidth of the fluors is set at 20 nm, where no bleed-through is expected; however, if needed, it can be adjusted to minimize bleed-through. The performance of the multiplexed AptaFluor sensor is tested in the presence of a mixture of NFs and compared to the non-multiplexed sensor (as discussed above) as well as commercially available ELISA kits. In addition, the specificity of the sensor against other NFs and signal stability can be determined as described above. Furthermore, the multiplexed AptaFluor assay can be used to screen the library of pharmacologically active compounds (LOPAC), which is a collection of 1280 pharmacologically active compounds frequently used for HTS assay validation to assess potential interference and compatibility with drug screening libraries (Yan, et al., 2002, Journal of biomolecular screening, 7(5):451-459; Minond, et al., 2009, Bioorganic & medicinal chemistry, 17(14):5027-5037).

Multiplexed detection of the 3 NFs with sensitivity <1 nM, S/B ratio ≥3, LLD ≤0.1 nM, and at-least 250-fold specificity as compared to other NFs. The sensor(s) stability is expected to be ≥24 hours at 37° C. in cell culture media. Because the excitation peaks of Tb—which serves as the excitation source for fluorophores—are narrow and sufficiently separated from each other, cross-excitation among the acceptor fluors is not expected. Acceptor fluorophores that exhibit minimal fluorescence bleed-through have been selected; however, it may be possible that the skewed asymmetric fluorophore spectra may still result in slight spectral bleed-through. If multiplexing using Tb-chelate alone is not achieved, a combination of 3 lanthanide complexes—Tb, Eu, and Sm, which have emission peaks at 545, 615, and 645 nm, respectively, can be used. The compatibility of using dual lanthanides for multiplexed assay has been demonstrated earlier (Zhang, et al, 2007, Chemistry of Materials, 19(24):5875-5881; Horton, et al., 2010, Journal of biomolecular screening, 15(8):1008-1015; Hilal, et al., 2010, Journal of biomolecular screening, 15(3):268-278; Rodriguez-Diaz, et al., 2003, Analytica chimica acta, 494(1):55-62), and the specific combination of terbium, europium, and samarium has previously been employed for multiplexed detection of caspases 1, 3 and 6 (Karvinen, et al., 2004, Analytical biochemistry, 325(2):317-325).

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention.

### REFERENCES

1 Loughlin S. E. and Fallon J. H., Neurotrophic factors. 2012: Elsevier.
2. Bartus R. T., Baumann T. L., Brown L, Kruegel B. R., Ostrove J. M., and Herzog C. D., Advancing neurotrophic factors as treatments for age-related neurodegenerative diseases: developing and demonstrating “clinical proof-of-concept” for AAV-neurturin (CERE-120) in Parkinson's disease. Neurobiology of aging, 2013. 34(1): p. 35-61.
3. Hefti F., Hartikka J., and Knusel B., Function of neurotrophic factors in the adult and aging brain and their possible use in the treatment of neurodegenerative diseases. Neurobiology of aging, 1989. 10(5): p. 515-533.
4. Kells A. P., Forsayeth J., and Bankiewicz K. S., Glial-derived neurotrophic factor gene transfer for Parkinson's disease: Anterograde distribution of AAV2 vectors in the primate brain. Neurobiology of disease, 2012. 48(2): p. 228-235.
5. Yasuda T. and Mochizuki H., Use of growth factors for the treatment of Parkinson's disease. Expert review of neurotherapeutics, 2010. 10(6): p. 915-924.
6. Emborg M., Moirano J., Raschke J., Bondarenko V., Zufferey R., Peng S., Ebert A., Joers V., Roitberg B., and Holden J., Response of aged parkinsonian monkeys to in vivo gene transfer of GDNF. Neurobiology of disease, 2009. 36(2): p. 303-311.
7. Zhao Y., Haney M. J., Gupta R., Bohnsack J. P., He Z., Kabanov A. V., and Batrakova E. V., GDNF-transfected macrophages produce potent neuroprotective effects in Parkinson's disease mouse model. PloS one, 2014. 9(9): p. e106867.
8. Eggert K., Schlegel J., Oertel W., Würz C., Krieg J-C., and Vedder H., Glial cell line-derived neurotrophic factor protects dopaminergic neurons from 6-hydroxydopamine toxicity in vitro. Neuroscience letters, 1999. 269(3): p. 178-182.
9. Hegarty S. V., Sullivan A. M., and O'Keeffe G. W., Roles for the TGF β superfamily in the development and survival of midbrain dopaminergic neurons. Molecular neurobiology, 2014. 50(2): p. 559-573.
10. Hegarty S. V., O'Keeffe G. W., and Sullivan A. M., Neurotrophic factors: from neurodevelopmental regulators to novel therapies for Parkinson's disease. Neural regeneration research, 2014. 9(19): p. 1708.
11. Nam J. H., Leem E., Jeon M-T., Jeong K. H., Park J-W., Jung U. J., Kholodilov N., Burke R. E., Jin B. K., and Kim S. R., Induction of GDNF and BDNF by hRheb (S16H) transduction of SNpc neurons: neuroprotective mechanisms of hRheb (S16H) in a model of Parkinson's disease. Molecular neurobiology, 2015. 51(2): p. 487-499.
12. Yang F., Feng L., Zheng F., Johnson S. W., Du J., Shen L., Wu C-P., and Lu B., GDNF acutely modulates excitability and A-type K+ channels in midbrain dopaminergic neurons. Nature neuroscience, 2001. 4(11): p. 1071-1078.
13. Hebert M. A., Van Horne C. G., Hoffer B. J., and Gerhardt G. A., Functional effects of GDNF in normal rat striatum: presynaptic studies using in vivo electrochemistry and microdialysis. Journal of Pharmacology and Experimental Therapeutics, 1996. 279(3): p. 1181-1190.
14. Peterson A. L. and Nutt J. G., Treatment of Parkinson's disease with trophic factors. Neurotherapeutics, 2008. 5(2): p. 270-280.
15. Rosenblad C., Kirk D., and Björklund A., Sequential administration of GDNF into the substantia nigra and striatum promotes dopamine neuron survival and axonal sprouting but not striatal reinnervation or functional recovery in the partial 6-OHDA lesion model. Experimental neurology, 2000. 161(2): p. 503-516.
16. Winkler C., Sauer H., Lee C. S., and Björklund A., Short-term GDNF treatment provides long-term rescue of lesioned nigral dopaminergic neurons in a rat model of Parkinson's disease. The Journal of neuroscience, 1996. 16(22): p. 7206-7215.
17. Sauer H. and Oertel W., Progressive degeneration of nigrostriatal dopamine neurons following intrastriatal terminal lesions with 6-hydroxydopamine: a combined retrograde tracing and immunocytochemical study in the rat. Neuroscience, 1994. 59(2): p. 401-415.
18. Yoshii A. and Constantine-Paton M., Postsynaptic BDNF-TrkB signaling in synapse maturation, plasticity, and disease. Developmental neurobiology, 2010. 70(5): p. 304-322.
19. Lu B., Nagappan G., Guan X., Nathan P. J., and Wren P., BDNF-based synaptic repair as a disease-modifying strategy for neurodegenerative diseases. Nature Reviews Neuroscience, 2013. 14(6): p. 401-416.
20. Selkoe D. J., Alzheimer's disease is a synaptic failure. Science, 2002. 298(5594): p. 789-791.
21. Terry R. D., Masliah E., Salmon D. P., Butters N., DeTeresa R., Hill R., Hansen L. A., and Katzman R., Physical basis of cognitive alterations in Alzheimer's disease: synapse loss is the major correlate of cognitive impairment. Annals of neurology, 1991. 30(4): p. 572-580.
22. Park H. and Poo M-M., Neurotrophin regulation of neural circuit development and function. Nature Reviews Neuroscience, 2013. 14(1): p. 7-23.
23. McAllister A. K., Lo D. C., and Katz L. C., Neurotrophins regulate dendritic growth in developing visual cortex. Neuron, 1995. 15(4): p. 791-803.
24. Cabelli R. J., Hohn A., and Shatz C. J., Inhibition of ocular dominance column formation by infusion of NT-4/5 or BDNF. Science, 1995. 267(5204): p. 1662.
25. Pozzo-Miller L. D., Gottschalk W., Zhang L., McDermott K., Du J., Gopalakrishnan R., Oho C., Sheng Z-H., and Lu B., Impairments in high-frequency transmission, synaptic vesicle docking, and synaptic protein distribution in the hippocampus of BDNF knockout mice. The Journal of Neuroscience, 1999. 19(12): p. 4972-4983.
26. Genoud C., Knott G. W., Sakata K., Lu B., and Welker E., Altered synapse formation in the adult somatosensory cortex of brain-derived neurotrophic factor heterozygote mice. The Journal of neuroscience, 2004. 24(10): p. 2394-2400.
27. Jovanovic J. N., Czernik A. J., Fienberg A. A., Greengard P., and Sihra T. S., Synapsins as mediators of BDNF-enhanced neurotransmitter release. Nature neuroscience, 2000. 3(4): p. 323-329.
28. Baquet Z. C., Bickford P. C., and Jones K. R., Brain-derived neurotrophic factor is required for the establishment of the proper number of dopaminergic neurons in the substantia nigra pars compacta. The Journal of neuroscience, 2005. 25(26): p. 6251-6259.
29. Howells D., Porritt M., Wong J., Batchelor P., Kalnins R., Hughes A., and Donnan G., Reduced BDNF mRNA expression in the Parkinson's disease substantia nigra. Experimental neurology, 2000. 166(1): p. 127-135.
30. Mogi M., Togari A., Kondo T., Mizuno Y., Komure O., Kuno S., Ichinose H., and Nagatsu T., Brain-derived growth factor and nerve growth factor concentrations are decreased in the substantia nigra in Parkinson's disease. Neuroscience letters, 1999. 270(1): p. 45-48.
31. Chen K. S., Nishimura M. C., Armanini M. P., Crowley C., Spencer S. D., and Phillips H. S., Disruption of a single allele of the nerve growth factor gene results in atrophy of basal forebrain cholinergic neurons and memory deficits. The Journal of neuroscience, 1997. 17(19): p. 7288-7296.
32. Weissmiller A. M. and Wu C., Current advances in using neurotrophic factors to treat neurodegenerative disorders. Translational neurodegeneration, 2012. 1(1): p. 1.
33. Cooper J. D., Salehi A., Delcroix J-D., Howe C. L., Belichenko P. V., Chua-Couzens J., Kilbridge J. F., Carlson E. J., Epstein C. J., and Mobley W. C., Failed retrograde transport of NGF in a mouse model of Down's syndrome: reversal of cholinergic neurodegenerative phenotypes following NGF infusion. Proceedings of the National Academy of Sciences, 2001. 98(18): p. 10439-10444.
34. Venero J., Knüsel B., Beck K., and Hefti F., Expression of neurotrophin and trk receptor genes in adult rats with fimbria transections: effect of intraventricular nerve growth factor and brain-derived neurotrophic factor administration. Neuroscience, 1994. 59(4): p. 797-815.
35. Massa S. M., Yang T., Xie Y., Shi J., Bilgen M., Joyce J. N., Nehama D., Rajadas J., and Longo F. M., Small molecule BDNF mimetics activate TrkB signaling and prevent neuronal degeneration in rodents. The Journal of clinical investigation, 2010. 120(5): p. 1774-1785.
36. Longo F. M., Yang T., Knowles J. K., Xie Y., Moore L. A., and Massa S. M., Small molecule neurotrophin receptor ligands: novel strategies for targeting Alzheimer's disease mechanisms. Current Alzheimer Research, 2007. 4(5): p. 503-506.
37. Sen S., Duman R., and Sanacora G., Serum brain-derived neurotrophic factor, depression, and antidepressant medications: meta-analyses and implications. Biological psychiatry, 2008. 64(6): p. 527-532.
38. Liu H-T. and Kuo H-C., Urinary nerve growth factor level could be a potential biomarker for diagnosis of overactive bladder. The Journal of urology, 2008. 179(6): p. 2270-2274.
39. Gale M. and Yan Q., High-throughput screening to identify inhibitors of lysine demethylases, 2015.
40. Eglen R. M., Reisine T., Roby P., Rouleau N., Illy C., Bossé R., and Bielefeld M., The use of AlphaScreen technology in HTS: current status. Current chemical genomics, 2008. 1: p. 2.
41. Pradidarcheep W., Labruyère W. T., Dabhoiwala N. F., and Lamers W. H., Lack of specificity of commercially available antisera: better specifications needed. Journal of Histochemistry & Cytochemistry, 2008. 56(12): p. 1099-1111.
42. Michel M. C., Wieland T., and Tsujimoto G., How reliable are G-protein-coupled receptor antibodies? Naunyn-Schmiedeberg's archives of pharmacology, 2009. 379(4): p. 385-388.
43. Bodei S., Arrighi N., Spano P., and Sigala S., Should we be cautious on the use of commercially available antibodies to dopamine receptors? Naunyn-Schmiedeberg's archives of pharmacology, 2009. 379(4): p. 413-415.
44. Hamdani N. and van der Velden J., Lack of specificity of antibodies directed against human beta-adrenergic receptors. Naunyn-Schmiedeberg's archives of pharmacology, 2009. 379(4): p. 403-407.
45. Jositsch G., Papadakis T., Haberberger R. V., Wolff M., Wess J., and Kummer W., Suitability of muscarinic acetylcholine receptor antibodies for immunohistochemistry evaluated on tissue sections of receptor gene-deficient mice. Naunyn-Schmiedeberg's archives of pharmacology, 2009. 379(4): p. 389-395.
46. Pradidarcheep W., Stallen J., Labruyère W. T., Dabhoiwala N. F., Michel M. C., and Lamers W. H., Lack of specificity of commercially available antisera against muscarinergic and adrenergic receptors. Naunyn-Schmiedeberg's archives of pharmacology, 2009. 379(4): p. 397-402.
47. Jensen B. C., Swigart P. M., and Simpson P. C., Ten commercial antibodies for alpha-1-adrenergic receptor subtypes are nonspecific. Naunyn-Schmiedeberg's archives of pharmacology, 2009. 379(4): p. 409-412.
48. Velcheti V., Schalper K. A., Carvajal D. E., Anagnostou V. K., Syrigos K. N., Sznol M., Herbst R. S., Gettinger S. N., Chen L., and Rimm D. L., Programmed death ligand-1 expression in non-small cell lung cancer. Laboratory investigation, 2014. 94(1): p. 107-116.
49. Tombelli S., Minunni M., and Mascini M., Analytical applications of aptamers. Biosensors and Bioelectronics, 2005. 20(12): p. 2424-2434
50. Stoltenburg R., Reinemann C., and Strehlitz B., SELEX—a (r) evolutionary method to generate high-affinity nucleic acid ligands. Biomolecular engineering, 2007. 24(4): p. 381-403.
51. Jayasena S. D., Aptamers: an emerging class of molecules that rival antibodies in diagnostics. Clinical chemistry, 1999. 45(9): p. 1628-1650.
52. McKeague M. and DeRosa M. C., Challenges and opportunities for small molecule aptamer development. Journal of nucleic acids, 2012. 2012.
53. Ernfors P., Ibanez C. F., Ebendal T., Olson L., and Persson H., Molecular cloning and neurotrophic activities of a protein with structural similarities to nerve growth factor: developmental and topographical expression in the brain. Proceedings of the National Academy of Sciences, 1990. 87(14): p. 5454-5458.
54. Li D., Song S., and Fan C., Target-responsive structural switching for nucleic acid-based sensors. Accounts of Chemical Research, 2010. 43(5): p. 631-641.
55. Nutiu R. and Li Y., Structure-switching signaling aptamers. Journal of the American Chemical Society, 2003. 125(16): p. 4771-4778.
56. Nutiu R. and Li Y., Aptamers with fluorescence-signaling properties. Methods, 2005. 37(1): p. 16-25.
57. Handl H. L. and Gillies R. J., Lanthanide-based luminescent assays for ligand-receptor interactions. Life sciences, 2005. 77(4): p. 361-371.
58. Freeman. R, Girsh J., Fang-ju Jou A., Ho J-aA., Hug T., Dernedde J., and Willner I., Optical aptasensors for the analysis of the vascular endothelial growth factor (VEGF). Analytical chemistry, 2012. 84(14): p. 6192-6198.
59. Oh S. S., Plakos K., Lou X., Xiao Y., and Soh H. T., In vitro selection of structure-switching, self-reporting aptamers. Proceedings of the National Academy of Sciences, 2010. 107(32): p. 14053-14058.
60. Zhang S., Xia J., and Li X., Electrochemical biosensor for detection of adenosine based on structure-switching aptamer and amplification with reporter probe DNA modified Au nanoparticles. Analytical chemistry, 2008. 80(22): p. 8382-8388.
61. Tang Z., Mallikaratchy P., Yang R., Kim Y., Zhu Z., Wang H., and Tan W., Aptamer switch probe based on intramolecular displacement. Journal of the American Chemical Society, 2008. 130(34): p. 11268-11269.
62. Tuleuova N., Jones C. N., Yan J., Ramanculov E., Yokobayashi Y., and Revzin A., Development of an aptamer beacon for detection of interferon-gamma. Analytical chemistry, 2010. 82(5): p. 1851-1857.
63. Lu Y., Li X., Zhang L., Yu P., Su L., and Mao L., Aptamer-based electrochemical sensors with aptamer-complementary DNA oligonucleotides as probe. Analytical chemistry, 2008. 80(6): p. 1883-1890.
64. Yan Y-X., Boldt-Houle D. M., Tillotson B. P., Gee M. A., Brian J., Chang X-J., Olesen C. E., and Palmer M. A., Cell-based high-throughput screening assay system for monitoring G protein-coupled receptor activation using β-galactosidase enzyme complementation technology. Journal of biomolecular screening, 2002. 7(5): p. 451-459.
65. Minond D., Saldanha S. A., Subramaniam P., Spaargaren M., Spicer T., Fotsing J. R., Weide T., Fokin V. V., Sharpless K. B., and Galleni M., Inhibitors of VIM-2 by screening pharmacologically active and click-chemistry compound libraries. Bioorganic & medicinal chemistry, 2009. 17(14): p. 5027-5037.
66. Zhang H., Xu Y., Yang W., and Li Q., Dual-lanthanide-chelated silica nanoparticles as labels for highly sensitive time-resolved fluorometry. Chemistry of Materials, 2007. 19(24): p. 5875-5881.
67. Horton R. A. and Vogel K. W., Multiplexing terbium- and europium-based TR-FRET readouts to increase kinase assay capacity. Journal of biomolecular screening, 2010. 15(8): p. 1008-1015.
68. Hilal T., Puetter V., Otto C., Parczyk K., and Bader B., A dual estrogen receptor TR-FRET assay for simultaneous measurement of steroid site binding and coactivator recruitment. Journal of biomolecular screening, 2010. 15(3): p. 268-278.
69. Rodriguez-Diaz R., Aguilar-Caballos M., and Gómez-Hens A., Simultaneous determination of ciprofloxacin and tetracycline in biological fluids based on dual-lanthanide sensitised luminescence using dry reagent chemical technology. Analytica chimica acta, 2003. 494(1): p. 55-62.
70. Karvinen J., Elomaa A., Mäkinen M-L., Hakala H., Mukkala V-M., Peuralahti J., Hurskainen P., Hovinen J., and Hemmilä I., Caspase multiplexing: simultaneous homogeneous time-resolved quenching assay (TruPoint) for caspases 1, 3, and 6. Analytical biochemistry, 2004. 325(2): p. 317-325.
71. Yüce M., Ullahb N., and Budak H., Trends in aptamer selection methods and Applications. Analyst, 2015. 140:5379-5399.

What is claimed is:

1. A sensor for measuring an analyte, comprising:
(a) an aptamer and
(b) an oligonucleotide;
wherein the oligonucleotide is complementary to the aptamer;
wherein the aptamer and the oligonucleotide are individually conjugated to a lanthanide donor or an organic fluor acceptor; and
wherein the aptamer and the oligonucleotide are dissociated in the presence of the analyte.

2. The sensor of claim 1, wherein:
(a) the analyte is an amino acid, an amino acid derivative, a peptide, a protein, a steroid, a lipid, a sugar, a carbohydrate, a drug molecule, a drug metabolite, a coenzyme, a nucleotide, a nucleotide derivative, a cyclic nucleotide, and/or a cyclic dinucleotide;
(b) the lanthanide donor is a terbium, europium, and/or samarium chelate;
(c) the organic fluor acceptor is Fluorescein, rhodamine, Texas Red, an Alexa Fluor, a Cyanine dye, and/or an Atto dye; and/or
(d) the organic fluor acceptor is a non-overlapping organic fluor acceptor.

3. A sensor for measuring an analyte, comprising:
(a) an aptamer and
(b) an oligonucleotide;
wherein the oligonucleotide is complementary to the aptamer;
wherein the aptamer and the oligonucleotide are individually conjugated to a lanthanide donor or an organic fluor acceptor; and
wherein the aptamer and the oligonucleotide are associated in the presence of the analyte.

4. The sensor of claim 3, wherein:
(a) the analyte is an amino acid, an amino acid derivative, a peptide, a protein, a steroid, a lipid, a sugar, a carbohydrate, a drug molecule, a drug metabolite, a coenzyme, a nucleotide, a nucleotide derivative, a cyclic nucleotide, and/or a cyclic dinucleotide;
(b) the lanthanide donor is a terbium, europium, and/or samarium chelate;
(c) the organic fluor acceptor is Fluorescein, rhodamine, Texas Red, an Alexa Fluor, a Cyanine dye, and/or an Atto dye; and/or
(d) the organic fluor acceptor is a non-overlapping organic fluor acceptor.

5. A sensor for measuring an analyte, comprising:
(a) a first aptamer specific for a first analyte;
(b) a second aptamer specific for a second analyte;
(c) a first oligonucleotide; and
(d) a second oligonucleotide;
wherein the first oligonucleotide is complementary to the first aptamer;
wherein the second oligonucleotide is complementary to the second aptamer;
wherein the first and the second aptamer and the first and the second oligonucleotide are each individually conjugated to a lanthanide donor or an organic fluor acceptor;
wherein the first aptamer and the first oligonucleotide are dissociated in the presence of the first analyte; and
wherein the second aptamer and the second oligonucleotide are dissociated in the presence of the second analyte.

6. The sensor of claim 5, wherein:
(a) the analyte is an amino acid, an amino acid derivative, a peptide, a protein, a steroid, a lipid, a sugar, a carbohydrate, a drug molecule, a drug metabolite, a coenzyme, a nucleotide, a nucleotide derivative, a cyclic nucleotide, and/or a cyclic dinucleotide;
(b) the lanthanide donor is a terbium, europium, and/or samarium chelate;
(c) the organic fluor acceptor is Fluorescein, rhodamine, Texas Red, an Alexa Fluor, a Cyanine dye, and/or an Atto dye; and/or
(d) the organic fluor acceptor is a non-overlapping organic fluor acceptor.

7. A sensor for measuring an analyte, comprising:
(a) a first aptamer specific for a first analyte;
(b) a second aptamer specific for a second analyte;
(c) a first oligonucleotide; and
(d) a second oligonucleotide;
wherein the first oligonucleotide is complementary to the first aptamer;
wherein the second oligonucleotide is complementary to the second aptamer;
wherein the first and the second aptamer and the first and the second oligonucleotide are each individually conjugated to a lanthanide donor or an organic fluor acceptor;
wherein the first aptamer and the first oligonucleotide are associated in the presence of the first analyte; and
wherein the second aptamer and the second oligonucleotide are associated in the presence of the second analyte.

8. The sensor of claim 7, wherein:
(a) the analyte is an amino acid, an amino acid derivative, a peptide, a protein, a steroid, a lipid, a sugar, a carbohydrate, a drug molecule, a drug metabolite, a coenzyme, a nucleotide, a nucleotide derivative, a cyclic nucleotide, and/or a cyclic dinucleotide;
(b) the lanthanide donor is a terbium, europium, and/or samarium chelate;
(c) the organic fluor acceptor is Fluorescein, rhodamine, Texas Red, an Alexa Fluor, a Cyanine dye, and/or an Atto dye; and/or
(d) the organic fluor acceptor is a non-overlapping organic fluor acceptor.

9. A method for measuring an analyte, comprising:
(a) contacting a sensor with an analyte;
wherein the sensor comprises an aptamer associated with a complementary oligonucleotide in the absence of the analyte;
wherein the aptamer and oligonucleotide are individually conjugated to a lanthanide donor or an organic fluor acceptor; and
wherein the aptamer and oligonucleotide disassemble in the presence of the analyte; and
(b) detecting a signal generated upon dissociation of the aptamer and oligonucleotide; thereby measuring the analyte.

10. The method of claim 9, wherein the signal is a time-resolved fluorescence energy transfer (TR-FRET) signal.

11. The method of claim 10, wherein:
(a) the analyte is an amino acid, an amino acid derivative, a peptide, a protein, a steroid, a lipid, a sugar, a carbohydrate, a drug molecule, a drug metabolite, a coenzyme, a nucleotide, a nucleotide derivative, a cyclic nucleotide, and/or a cyclic dinucleotide;
(b) the lanthanide donor is a terbium, europium, and/or samarium chelate;
(c) the organic fluor acceptor is Fluorescein, rhodamine, Texas Red, an Alexa Fluor, a Cyanine dye, and/or an Atto dye; and/or
(d) the organic fluor acceptor is a non-overlapping organic fluor acceptor.

12. A method for measuring an analyte, comprising:
(a) contacting a sensor with an analyte;
wherein the sensor comprises an aptamer and an oligonucleotide;
wherein the oligonucleotide is complementary to the aptamer;
wherein the aptamer and oligonucleotide are individually conjugated to a lanthanide donor or an organic fluor acceptor; and
wherein the aptamer and oligonucleotide assemble in the presence of the analyte to form a trimeric complex comprising the aptamer, the oligonucleotide, and the analyte; and
(b) detecting a signal generated upon assembly of the trimeric complex; thereby measuring the analyte.

13. The method of claim 12, wherein the signal is a time-resolved fluorescence energy transfer (TR-FRET) signal.

14. The method of claim 12, wherein:
(a) the analyte is an amino acid, an amino acid derivative, a peptide, a protein, a steroid, a lipid, a sugar, a carbohydrate, a drug molecule, a drug metabolite, a coenzyme, a nucleotide, a nucleotide derivative, a cyclic nucleotide, and/or a cyclic dinucleotide;
(b) the lanthanide donor is a terbium, europium, and/or samarium chelate;
(c) the organic fluor acceptor is Fluorescein, rhodamine, Texas Red, an Alexa Fluor, a Cyanine dye, and/or an Atto dye; and/or
(d) the organic fluor acceptor is a non-overlapping organic fluor acceptor.

15. A method for measuring an analyte, comprising:
(a) contacting a sensor with an analyte;
wherein the sensor comprises a first aptamer specific for a first analyte associated with a first complementary oligonucleotide in the absence of the first analyte, and a second aptamer specific for a second analyte associated with a second complementary oligonucleotide in the absence of the second analyte;
wherein the first and the second aptamer and the first and the second oligonucleotide are each individually conjugated to a lanthanide donor or an organic fluor acceptor;
wherein the first aptamer and the first oligonucleotide disassemble in the presence of the first analyte;
wherein the second aptamer and the second oligonucleotide disassemble in the presence of the second analyte; and
(b) detecting a first signal generated upon dissociation of the first aptamer and the first oligonucleotide; thereby measuring the first analyte; and
(c) detecting a second signal generated upon dissociation of the second aptamer and the second oligonucleotide; thereby measuring the second analyte.

16. The method of claim 15, wherein the signal is a time-resolved fluorescence energy transfer (TR-FRET) signal.

17. The method of claim 15, wherein:
(a) the analyte is an amino acid, an amino acid derivative, a peptide, a protein, a steroid, a lipid, a sugar, a carbohydrate, a drug molecule, a drug metabolite, a coenzyme, a nucleotide, a nucleotide derivative, a cyclic nucleotide, and/or a cyclic dinucleotide;
(b) the lanthanide donor is a terbium, europium, and/or samarium chelate;
(c) the organic fluor acceptor is Fluorescein, rhodamine, Texas Red, an Alexa Fluor, a Cyanine dye, and/or an Atto dye; and/or
(d) the organic fluor acceptor is a non-overlapping organic fluor acceptor.

18. A method for measuring an analyte, comprising:
(a) contacting a sensor with an analyte;
wherein the sensor comprises:
(i) a first aptamer specific for a first analyte;
(ii) a second aptamer specific for a second analyte;
(iii) a first oligonucleotide; and
(iv) a second oligonucleotide;
wherein the first oligonucleotide is complementary to the first aptamer;
wherein the second oligonucleotide is complementary to the second aptamer;
wherein the first and the second aptamer and the first and the second oligonucleotide are each individually conjugated to a lanthanide donor or an organic fluor acceptor;
wherein the first aptamer and the first oligonucleotide assemble in the presence of the first analyte to form a first trimeric complex comprising the first aptamer, the first oligonucleotide, and the first analyte;
wherein the second aptamer and the second oligonucleotide assemble in the presence of the second analyte to form a second trimeric complex comprising the second aptamer, the second oligonucleotide, and the second analyte;
(b) detecting a first signal generated upon assembly of the first trimeric complex; thereby measuring the first analyte; and
(c) detecting a second signal generated upon assembly of the second trimeric complex; thereby measuring the second analyte.

19. The method of claim 18, wherein the signal is a time-resolved fluorescence energy transfer (TR-FRET) signal.

20. The method of claim 18, wherein:
(a) the analyte is an amino acid, an amino acid derivative, a peptide, a protein, a steroid, a lipid, a sugar, a carbohydrate, a drug molecule, a drug metabolite, a coenzyme, a nucleotide, a nucleotide derivative, a cyclic nucleotide, and/or a cyclic dinucleotide;
(b) the lanthanide donor is a terbium, europium, and/or samarium chelate;
(c) the organic fluor acceptor is Fluorescein, rhodamine, Texas Red, an Alexa Fluor, a Cyanine dye, and/or an Atto dye; and/or
(d) the organic fluor acceptor is a non-overlapping organic fluor acceptor.
